(19) United States
(12) Patent Application Publication
(10) Pub. No.: US 2023/0367990 A1
(43) Pub. Date: Nov. 16, 2023

(54) SYSTEMS AND METHODS OF USING SPIKE TRAINS

(71) Applicant: University of Pittsburgh-Of the Commonwealth System of Higher Education, Pittsburgh, PA (US)

(72) Inventors: Benjamin Ryad Benosman, Pittsburgh, PA (US); Andrew B. Schwartz, Pittsburgh, PA (US); Himanshu Akolkar, Pittsburgh, PA (US)

(73) Assignee: University of Pittsburgh-Of the Commonwealth System of Higher Education, Pittsburgh, PA (US)

(21) Appl. No.: 18/027,902

(22) PCT Filed: Sep. 24, 2021
(86) PCT No.: PCT/US21/51950

§ 371 Date: Mar. 22, 2023

Related U.S. Application Data
(60) Provisional application No. 63/083,482, filed on Sep. 25, 2020.

(51) Int. Cl. G06N 3/02; G06F 3/01

(52) U.S. Cl. CPC G06N 3/02; G06F 3/015

(57) ABSTRACT

Disclosed herein, in certain embodiments, are methods and systems for utilizing spike trains. In one aspect, encompassed by the disclosure is a method comprising: determining, by at least one processor, a plurality of spike trains from a plurality of neurons of a cerebral cortex of a subject. The method may comprise determining, by the at least one processor, a plurality of first layer feature vectors, the plurality of first layer feature vectors including a first feature vector that comprises temporal-relation values of a subset of the plurality of spike trains with respect to a time instance of a reference spike train of the subset, corresponding to a time constant τ having a first value. The method may comprise establishing, by the at least one processor, a first layer comprising a first plurality of clusters of the first layer feature vectors.

This application is related to and claims priority under 35 U.S. § 119(e) the U.S. Provisional Patent Application No. 63/083,482, filed Sep. 25, 2020, titled “SYSTEMS AND METHODS OF USING SPIKE TRAINS,” the entire contents of which are incorporated herein by reference for all purposes.

### FIELD OF DISCLOSURE

The present disclosure is generally related to use of neural signals, including but not limited to devices, systems and methods of using spike trains.

### BACKGROUND

Developments of remote-controlled devices and extended reality immersive technologies are complex technological fields. Devices such as prosthetics and mind-controlled robotics should be engineered to be mechanically capable of accomplishing the required task, but also incorporate or operate with a system which can determine a user's intent for the specific task. Similarly, immersive technologies should capture a user's intended action for application by an avatar within a virtual reality (VR), augmented reality (AR), or mixed reality environment. Decoding a user's intended action and implementing such action remain an area to be developed.

### SUMMARY

In certain embodiments, disclosed herein are systems, methods, and non-transitory computer readable medium for utilizing spike trains for decoding a subject's intended motion. In one aspect, encompassed by the disclosure is a method comprising: determining, by at least one processor, a plurality of spike trains from a plurality of neurons of a cerebral cortex of a subject. The method may comprise determining, by the at least one processor, a plurality of first layer feature vectors, the plurality of first layer feature vectors including a first feature vector that comprises temporal-relation values of a subset of the plurality of spike trains with respect to a time instance of a reference spike train of the subset, corresponding to a time constant τ having a first value. The method may comprise establishing, by the at least one processor, a first layer comprising a first plurality of clusters of the first layer feature vectors. The method may comprise identifying, by the at least one processor, a set of one or more clusters from the first plurality of clusters most similar to a cluster formed according to an incoming feature vector. The method may comprise determining, by the at least one processor, a plurality of second layer feature vectors comprising a second plurality of clusters that includes the set of one or more clusters, the second plurality of clusters including a first cluster that comprises temporal-relation values of a cluster-subset of the second plurality of clusters with respect to a time instance of a reference cluster of the cluster-subset, corresponding to τ having a second value. The method may comprise establishing, by the at least one processor, a second layer comprising a first plurality of clusters of the second layer feature vectors. The method may comprise establishing, by the at least one processor, at least one successive layer after the second layer, each of the at least one successive layer corresponding to τ having a respective value. The method may comprise determining, by the at least one processor, that a type of motion performed by the subject corresponds to one or more clusters of layer feature vectors of one of the at least one successive layer or the second layer.

In some embodiments, the method may comprise detecting, by the at least one processor, a set of spike trains from the plurality of neurons. The method may comprise determining, by the at least one processor, that a first cluster based on the set of spike trains is consistent with the one or more clusters of layer feature vectors. The method may comprise determining, by the at least one processor responsive to determining that the first cluster is consistent with the one or more clusters of layer feature vectors, that the subject is intending or implementing the type of motion.

In certain embodiments, the second value may be larger than, smaller than, or equal to the first value. The second value may be larger than the first values, and each of the at least one successive layer corresponds to τ having a successively larger value than the second value. The τ for each of the at least one successive layer may be set at fixed intervals (e.g., intervals of 50 msec) or variable intervals (e.g., increasing/decreasing intervals). The τ for each of the at least one successive layer may correspond to a value of about 60 msec, about 70 msec, about 80 msec, about 90 msec, about 100 msec, about 110 msec, about 120 msec, about 130 msec, about 140 msec, about 150 msec, about 160 msec, about 170 msec, about 180 msec, about 190 msec, about 200 msec, or longer than 200 msec.

Each of the temporal-relation values of the first feature vector may be indicative of a difference in corresponding spike time with respect to the time instance of the reference spike train of the subset. The method may comprise determining, by the at least one processor, each of the temporal-relation values of the first feature vector according to: T=exp ((−|tᵢ−tⱼ|)/τ), wherein: tᵢ is the time instance of the reference spike train, tⱼ is a corresponding spike time of a neuron occurring immediately prior to the time instance of the reference spike train, τ is the time constant having the first value, and T is the corresponding temporal-relation value. The first value may be a value from about 5 msec to about 80 msec. The first value may be about 60 msec.

Each of the temporal-relation values of the first cluster may be indicative of a difference in corresponding spike time with respect to the time instance of the reference spike train of the cluster-subset. The method may comprise determining, by the at least one processor, each of the temporal-relation values of the first cluster of the second plurality of clusters according to: T=exp ((−|tₘ−tⱼ|)/τ), wherein: tₘ is the time instance of the reference cluster, tⱼ is a corresponding spike time of a neuron occurring immediately prior to the time instance of the reference cluster, τ is the time constant having the second value, and T is the corresponding temporal-relation value. The second value may be from about 60 to about 150 msec. The second value may be about 120 msec.

In some embodiments, the first plurality of clusters and the second plurality of clusters may be each independently determined by an unsupervised clustering algorithm. The unsupervised clustering algorithm may comprise a k-means clustering algorithm, a fuzzy k-means clustering algorithm, a hierarchical clustering, or a Gaussians mixture model (GMM).

The incoming feature vector may comprise a set of temporal-relation values, wherein each temporal-relation value from the set is determined according to: T=exp ((−|tₙ−tⱼ|)/τ), wherein: tₙ is a time instance of an additional reference spike train, tⱼ is a corresponding spike time of a neuron occurring immediately prior to the time instance of the additional reference spike train, τ is the first value, and T is the corresponding temporal-relation value.

In certain embodiments, the method may further comprise identifying, by the at least one processor, another set of one or more clusters from the first plurality of clusters of the second layer feature vectors most similar to a cluster formed according to a second incoming feature vector. The method may comprise determining, by the at least one processor, a plurality of third layer feature vectors comprising a third plurality of clusters that includes the another set of one or more clusters, the third plurality of clusters including a first cluster that comprises temporal-relation values of a cluster-subset of the third plurality of clusters with respect to a time instance of a reference cluster of the cluster-subset of the third plurality of clusters, corresponding to τ having a third value. The method may comprise establishing, by the at least one processor, a third layer comprising a first plurality of clusters of the third layer feature vectors. The method may comprise determining, by the at least one processor, each of the temporal-relation values of the first cluster of the third plurality of clusters according to: T=exp ((−|tₒ−tⱼ|)/τ), wherein: tₒ is the time instance of the reference cluster of the cluster-subset of the third plurality of clusters, tⱼ is a corresponding spike time of a neuron occurring immediately prior to the time instance of the reference cluster of the cluster-subset of the third plurality of clusters, τ is the time constant having the third value, and T is the corresponding temporal-relation value. The third value may be from about 110 to about 180 msec. The third value may be about 150 msec.

The first cluster may be determined to be consistent with the one or more clusters of layer feature vectors by a supervised learning method. The supervised learning method may comprise using a perceptron. The subject may be a human.

The disclosure also encompasses, in certain aspects, a system comprising at least one processor configured to determine a plurality of spike trains from a plurality of neurons of a cerebral cortex of a subject. The at least one processor can be configured to determine a plurality of first layer feature vectors, the plurality of first layer feature vectors including a first feature vector that comprises temporal-relation values of a subset of the plurality of spike trains with respect to a time instance of a reference spike train of the subset, corresponding to a time constant τ having a first value. The at least one processor can be configured to establish a first layer comprising a first plurality of clusters of the first layer feature vectors. The at least one processor can be configured to identify a set of one or more clusters from the first plurality of clusters most similar to a cluster formed according to an incoming feature vector. The at least one processor can be configured to determine a plurality of second layer feature vectors comprising a second plurality of clusters that includes the set of one or more clusters, the second plurality of clusters including a first cluster that comprises temporal-relation values of a cluster-sub set of the second plurality of clusters with respect to a time instance of a reference cluster of the cluster-subset, corresponding to τ having a second value. The at least one processor can be configured to establish a second layer comprising a first plurality of clusters of the second layer feature vectors. The at least one processor can be configured to establish at least one successive layer after the second layer, each of the at least one successive layer corresponding to τ having a respective value. The at least one processor can be configured to determine that a type of motion performed by the subject corresponds to one or more clusters of layer feature vectors of one of the at least one successive layer or the second layer.

In some embodiments, the at least one processor can be configured to detect a set of spike trains from the plurality neurons. The at least one processor can be configured to determine a first cluster based on the set of spike trains is consistent with the one or more clusters of layer feature vectors. The at least one processor can be configured to determine whether the subject is intending or implementing the type of motion by determining that the first cluster is consistent with the one or more clusters of layer feature vectors. The first cluster can be determined to be consistent with the one or more clusters of layer feature vectors by a supervised learning method, optionally determined by a perceptron.

The second value can be larger than, smaller than, or equal to the first value. The second value can be larger than the first values, and each of the at least one successive layer corresponds to T having a successively larger value than the second value.

In certain embodiments, each of the temporal-relation values of the first feature vector can be indicative of a difference in corresponding spike time with respect to the time instance of the reference spike train of the subset. The at least one processor can be configured to determine each of the temporal-relation values of the first feature vector according to: T=exp ((−|tᵢ−tⱼ|)/τ), wherein: tᵢ is the time instance of the reference spike train, tⱼ is a corresponding spike time of a neuron occurring immediately prior to the time instance of the reference spike train, τ is the time constant having the first value, and T is the corresponding temporal-relation value. The first value can be a value from about 5 msec to about 80 msec; or about 50 msec.

Each of the temporal-relation values of the first cluster can be indicative of a difference in corresponding spike time with respect to the time instance of the reference spike train of the cluster-subset. The at least one processor can be configured to determine each of the temporal-relation values of the first cluster of the second plurality of clusters according to: T=exp ((−|tₘ−tⱼ|)/τ), wherein: tₘ is the time instance of the reference cluster, tⱼ is a corresponding spike time of a neuron occurring immediately prior to the time instance of the reference cluster, τ is the time constant having the second value, and T is the corresponding temporal-relation value. The second value can be from about 60 to about 150 msec; or about 100 msec.

In some embodiments, the first plurality of clusters and the second plurality of clusters can each be independently determined by an unsupervised clustering algorithm. The unsupervised clustering algorithm can comprise a k-means clustering algorithm, a fuzzy k-means clustering algorithm, a hierarchical clustering, or a Gaussians mixture model (GMM).

The incoming feature vector can comprise a set of temporal-relation values, wherein each temporal-relation value from the set is determined according to: T=exp ((−|tₙ−tⱼ|)/τ), wherein: tₙ is a time instance of an additional reference spike train, tⱼ is a corresponding spike time of a neuron occurring immediately prior to the time instance of the additional reference spike train, τ is the first value, and T is the corresponding temporal-relation value. The τ for each of the at least one successive layer can correspond to a value of about 60 msec, about 70 msec, about 80 msec, about 90 msec, about 100 msec, about 110 msec, about 120 msec, about 130 msec, about 140 msec, about 150 msec, about 160 msec, about 170 msec, about 180 msec, about 190 msec, about 200 msec, or longer than 200 msec.

In certain embodiments, the at least one processor can further be configured to identifying another set of one or more clusters from the first plurality of clusters of the second layer feature vectors most similar to a cluster formed according to a second incoming feature vector. The at least one processor can be configured to determining a plurality of third layer feature vectors comprising a third plurality of clusters that includes the another set of one or more clusters, the third plurality of clusters including a first cluster that comprises temporal-relation values of a cluster-subset of the third plurality of clusters with respect to a time instance of a reference cluster of the cluster-subset of the third plurality of clusters, corresponding to τ having a third value. The at least one processor can be configured to establish a third layer comprising a first plurality of clusters of the third layer feature vectors. The at least one processor can be configured to determine each of the temporal-relation values of the first cluster of the third plurality of clusters according to: T=exp ((−|tₒ−tⱼ|)/τ), wherein: to is the time instance of the reference cluster of the cluster-subset of the third plurality of clusters, tⱼ is a corresponding spike time of a neuron occurring immediately prior to the time instance of the reference cluster of the cluster-subset of the third plurality of clusters, τ is the time constant having the third value, and T is the corresponding temporal-relation value.

The at least one processor (e.g., distributed across one or more systems/locations/devices) can be configured to wirelessly receive communication from an implanted device comprising one or more electrodes for detecting the plurality of spike trains. The at least one processor can be continuously updated by the implanted device.

The disclosure further encompasses, in some aspects, a non-transitory computer-readable medium (e.g., distributed or centralized storage medium/system) storing a program including instructions that, when executed by a processor, determines a plurality of spike trains from a plurality of neurons of a cerebral cortex of a subject. The program, when executed by the processor, can determine a plurality of first layer feature vectors, the plurality of first layer feature vectors including a first feature vector that comprises temporal-relation values of a subset of the plurality of spike trains with respect to a time instance of a reference spike train of the subset, corresponding to a time constant τ having a first value. The program, when executed by the processor, can establish a first layer comprising a first plurality of clusters of the first layer feature vectors. The program, when executed by the processor, can identify a set of one or more clusters from the first plurality of clusters most similar to a cluster formed according to an incoming feature vector. The program, when executed by the processor, can determine a plurality of second layer feature vectors comprising a second plurality of clusters that includes the set of one or more clusters, the second plurality of clusters including a first cluster that comprises temporal-relation values of a cluster-subset of the second plurality of clusters with respect to a time instance of a reference cluster of the cluster-subset, corresponding to τ having a second value. The program, when executed by the processor, can establish a second layer comprising a first plurality of clusters of the second layer feature vectors. The program, when executed by the processor, can establish at least one successive layer after the second layer, each of the at least one successive layer corresponding to τ having a respective value. The program, when executed by the processor, can determine that a type of motion performed by the subject corresponds to one or more clusters of layer feature vectors of one of the at least one successive layer or the second layer. The non-program, when executed by the processor, can detect a set of spike trains from the plurality neurons; determine a first cluster based on the set of spike trains is consistent with the one or more clusters of layer feature vectors; and determine whether the subject is intending or implementing the type of motion by determining that the first cluster is consistent with the one or more clusters of layer feature vectors.

Both the foregoing summary and the following description of the drawings and detailed description are exemplary and explanatory. They are intended to provide further details of the disclosure, but are not to be construed as limiting. Other objects, advantages, and novel features will be readily apparent to those skilled in the art from the following detailed description of the disclosure.

### BRIEF DESCRIPTION OF THE DRAWINGS

FIG. 1 is a flow chart illustrating a method disclosed herein for determining a subject's intended motion, according to one or more disclosed embodiments.

FIG. 2 is a block diagram of a system for use with a method described herein, according to one or more disclosed embodiments.

FIG. 3A shows examples of hand motions of a subject, according to one or more disclosed embodiments.

FIG. 3B shows neural spike train for an example trial of the vertical hand movement for the reaching part of the trial, according to one or more disclosed embodiments.

FIG. 3C shows an example of the temporal feature generation from each spike, according to one or more disclosed embodiments.

FIG. 3D shows 9 example clusters based on the temporal features generated from FIG. 3C, according to one or more disclosed embodiments.

FIG. 3E shows the correlation of the clusters with the actual hand motion of FIG. 3A, according to one or more disclosed embodiments, denoted by the corresponding symbols from FIG. 3D.

FIG. 4A shows hand trajectories for an example trail/path of motion, and activated clusters calculated in 3 successive layers, according to one or more disclosed embodiments. Each subsequent layer with longer timescale leads to stronger causal connections.

FIGS. 4B-4C show the results of decoding the activated clusters in a 2-dimensional space and in individual x and y directions, according to one or more disclosed embodiments.

FIG. 5A-FIG. 5D illustrate temporal patterns corresponding to hand movements in repeated target reach. FIG. 5B shows causal connection between different clusters. FIG. 5C shows zoomed in cluster activations for positions close to the target location. FIG. 5D The clusters have strong causal connections within subset of clusters from FIG. 5B.

FIG. 6 is a flow chart illustrating a process of determining a subject's intended motion, according to one or more disclosed embodiments.

FIG. 7 is a block diagram of a computing environment according to an example implementation of the present disclosure.

### DETAILED DESCRIPTION

### Overview

Neurons in the primary motor cortex (M1) can encode physical parameters of limb movements such as direction, force, extend, load, or posture. Such physical parameters are proposed to be encoded by the spikes and spike rates. In the visual and somatosensory cortex, spike times can carry significant information. Data from visual sensory cortex show highly precise response of neurons to repeating stimuli across the hierarchy of visual processing from retina, to Lateral Geniculate Nucleus (LGN) up to the higher visual areas and can be considered as temporal coding in the visual cortex. Similar temporal dynamics can be found in the auditory cortex′ vestibular system′, and somatosensory cortex.

In accordance with some of the inventive aspects disclosed herein, the motor cortex can be seen as similar or analogous to the somatosensory cortex in that it is in the same direct contact with the world as the somatosensory system is. For instance, the limb motion is expected to be associated with the same precise neural response as the neural response of a skin or joint encoding neuron would be expected to have.

The population dynamics of the neural spike trains can be based on firing rates or can consider neural activity at individual spike level. However, there are multiple problems with representing temporal dynamics of population of neurons. For example, it may be difficult to parameterize the time scale at which the temporal dynamics should be looked at, especially when applied to individual neurons. Further, there is no method to understand how different temporal patterns can combine with one another.

In some embodiments, provided herein are systems, methods, and non-transitory computer readable medium that take into account the temporal dynamics of a population of neurons and provide unique temporal patterns based on the temporal dynamics of the population of neurons. In some embodiments, the systems, methods, and non-transitory computer readable medium disclosed herein convert temporal delays between spikes coming from different units into a normalized feature map. This temporal map can then be used to represent and reveal unique spike patterns that occur repeatedly, or that can be associated with certain motions. In some instances, the temporal features are clustered into representative features that can be used to regenerate new spike trains encoding original spike patterns. In some instances, the new spike trains can be remapped into temporal features and clustered to reveal combinations of spike patterns. In some cases, the systems, methods, and non-transitory computer readable medium disclosed herein allows for these steps to be performed repeatedly multiple times to reveal spike patterns occurring over long time scales. In some cases, the systems, methods, and non-transitory computer readable medium disclosed herein allows for decoding the spike patterns and translating the spike patterns into a subject's intended motion. An intended motion may be defined and/or implemented by (or correlated with) neural activity (e.g., intended by a subject or as a consequence of involuntary/reflex action) such that a motion is intended or being willed, being performed, and/or is to be performed.

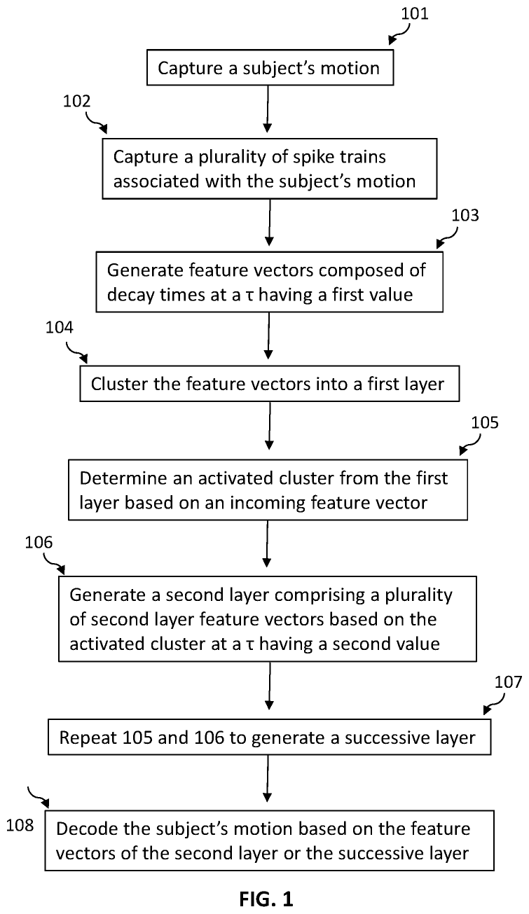

In certain embodiments, a method for decoding the subject's intended motion is as illustrated in FIG. 1. As shown in FIG. 1, a subject's motion (101) and a plurality of spike trains associated with the motion (102) are recorded (e.g., captured, measured, acquired). A reference spike train may be selected, and feature vectors composed of decay times, which encode a direction and magnitude, are calculated based on the reference spike train at a time constant τ having a first value (103). The feature vectors can then be clustered into a first layer comprising a first plurality of clusters (104). An incoming spike train can be recorded to generate an incoming feature vector. The incoming feature vector can then be compared to the first layer to determine the closest cluster to the incoming feature vector. The closest cluster to the incoming feature vector can be termed an activated cluster (105). The activated cluster can be selected as the reference for generating a plurality of second layer feature vectors at a time constant τ having a second value. The plurality of second layer feature vectors can further be clustered to generate a second layer comprising a second plurality of clusters (106). 105 and 106 can be optionally repeated to generate at least one successive layer (107). The subject's motion can be decoded based on the feature vectors of the second layer or the successive layer (108).

### Systems and Non-Transitory Computer-Readable Medium for Processing Spike Trains

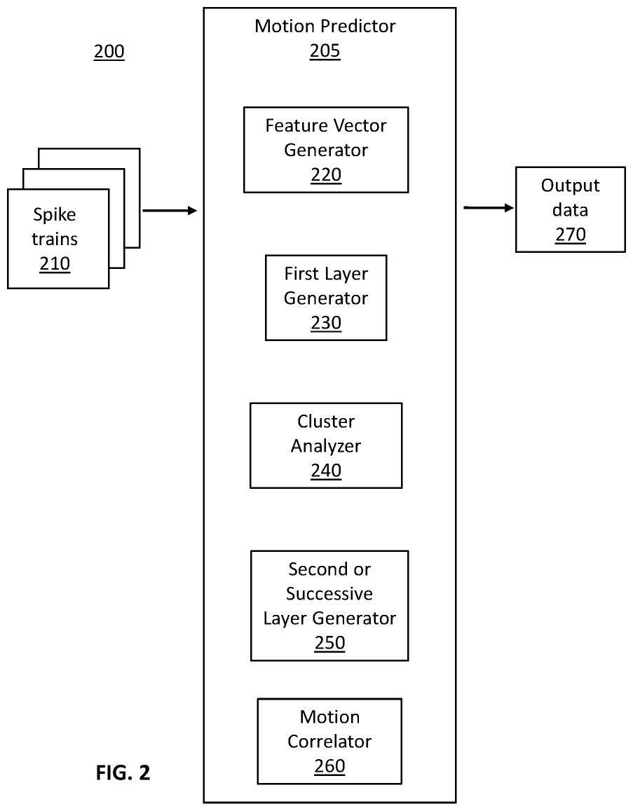

In certain embodiments, disclosed herein is a system for use with one or more of the methods described herein. FIG. 2 is a block diagram of a system 200 for predicting a motion of a subject, according to an example implementation of the present disclosure. The system 200 can include a motion predictor 205 which can further include a feature vector generator 220, a first layer generator 230, a cluster analyzer 240, a second or successive layer generator 250, and/or a motion correlator 260. One or more of these components may operate together to receive a plurality of spike trains 210 recorded from a subject and can subsequently generate an output data 270. Each of the component disclosed herein is implemented on a hardware, or a combination of software and hardware. For example, one or more of the components can be implemented as software modules executing on one or more processors. For another example, one or more of the components can be implemented as/in one or more hardware components such as a neural network chip, a field programmable gate logic (FPGA) and/or an application specific integrated circuit (ASIC). In some embodiments, the system 200 includes more, fewer, or different components than shown in FIG. 2.

The feature vector generator 220 includes or corresponds to a component that receives the plurality of spike trains 210. The plurality of spike trains 210 can be generated or recorded from a plurality of neurons of a cerebral cortex of a subject. In some instances, the feature vector generator 220 receives the plurality of spike trains 210 in real-time (e.g., from an implanted or wearable detector/measurement device on a subject). In other instances, the feature vector generator 220 receives the plurality of spike trains 210 at a predetermined time (e.g., in batch, as recorded and/or stored by the detector/measurement device), e.g., at about 10 minutes, 20 minutes, 30 minutes, 60 minutes, 2 hours, 3 hours, 4 hours, 5 hours, 6 hours, 12 hours, 24 hours, or more; intermittently, or based on user input. In additional instances, the feature vector generator 220 detects, measures, or records the plurality of spike trains 210, e.g., directly or indirectly from a subject.

As used herein, the terms “spike train” or “neural spike train” refer to a sequence of spikes or action potentials with respect to time. Spikes, also known as action potentials, refers to a change in the voltage across a neuron membrane due to the flow of ions into and out of the neuron. In some instances, an action potential begins as a result of depolarization. During depolarization, voltage gated sodium ion channels open due to an electrical stimulus. As the sodium enters into the cell, the positive sodium ions raise the charge inside the cell from negative to positive. Once a threshold is reached, an action potential is produced. The action potential then concludes with a refractory period in which the sodium channel closes and enters an inactive state. Each spike or action potential is considered as starting or being initiated at a single point in time. In some cases, each spike or action potential is correlated with a motion of a subject from which the spike or action potential is recorded.

The feature vector generator 220 can process, analyze, or otherwise determine a plurality of first layer feature vectors. The plurality of first layer feature vectors can include a first feature vector that comprises temporal-relation values of a subset of the plurality of spike trains. The temporal-relation values represent time-based, temporal relationships between spikes, obtained by translation/transformation of temporal relationships to fit into a finite interval or range (e.g., an interval of 0-1). The temporal-relation values can be processed, analyzed, or otherwise determined from the plurality of spike trains 210 with respect to a time instance of a reference spike train of the subset. A spike train of a single neuron from the plurality of spike trains 210 can be designated as the reference spike train. The time instance can correspond to a time constant τ having a first value (e.g., which can be different from other values or time constants associated with other layers).

Each of the temporal-relation values of the first feature vector can indicate a difference in corresponding spike time with respect to the time instance of the reference spike train of the subset. The difference can be analyzed, calculated, or otherwise determined by any algorithms or equations that can process the temporal relationships from the plurality of spike times to fit a finite interval or range. In one instance, the difference is determined by an increasing/decreasing function. The difference can vary with time, e.g., at a fixed or variable rate/interval. The function can be increasing (or non-decreasing) or can be strictly increasing. The function can be decreasing (or non-increasing) or can be strictly decreasing. The difference can be characterized with exponential growth/decay.

In one example implementation, the difference is determined by a decreasing or monotonic function. The difference can vary at a fixed or a variable rate/interval. The function can be decreasing (or non-increasing) or can be strictly decreasing. The difference can also be characterized with exponential decay or other types of decay or decrease. In some cases, each of the temporal-relation values of the first feature vector is determined according to Equation 1:

T=exp((−|tᵢ−tⱼ|)/τ)

in which tᵢ is the time instance of the reference spike train, tⱼ is a corresponding spike time of a neuron occurring immediately prior to the time instance of the reference spike train, τ is the time constant having the first value, and T is the corresponding temporal-relation value.

The first value can be any value greater than 1 msec for instance, and can be up to 500000 msec for instance, according to various implementations. The first value can be a value from about 5 msec to about 80 msec. The first value can be a value from about 5 msec to about 75 msec, from about 5 msec to about 70 msec, from about 5 msec to about 65 msec, from about 5 msec to about 60 msec, from about 5 msec to about 55 msec, from about 5 msec to about 50 msec, from about 5 msec to about 45 msec, from about 5 msec to about 40 msec, from about 5 msec to about 35 msec, from about 5 msec to about 30 msec, from about 5 msec to about 25 msec, from about 5 msec to about 20 msec, from about 10 msec to about 70 msec, from about 10 msec to about 65 msec, from about 10 msec to about 60 msec, from about 10 msec to about 55 msec, from about 10 msec to about 50 msec, from about 10 msec to about 45 msec, from about 10 msec to about 40 msec, from about 10 msec to about 35 msec, from about 10 msec to about 30 msec, from about 20 msec to about 70 msec, from about 20 msec to about 65 msec, from about 20 msec to about 60 msec, from about 20 msec to about 55 msec, from about 20 msec to about 50 msec, from about 20 msec to about 45 msec, from about 20 msec to about 40 msec, from about 20 msec to about 35 msec, from about 30 msec to about 70 msec, from about 30 msec to about 65 msec, from about 30 msec to about 60 msec, from about 30 msec to about 55 msec, from about 30 msec to about 50 msec, from about 30 msec to about 45 msec, from about 30 msec to about 40 msec, from about 40 msec to about 70 msec, from about 40 msec to about 65 msec, from about 40 msec to about 60 msec, from about 40 msec to about 55 msec, from about 40 msec to about 50 msec, from about 50 msec to about 70 msec, from about 50 msec to about 65 msec, or from about 50 msec to about 60 msec. The first value can be about 5 msec, 10 msec, 15 msec, 20 msec, 25 msec, 30 msec, 35 msec, 40 msec, 45 msec, 50 msec, 55 msec, 60 msec, 65 msec, 70 msec, 75 msec, or 80 msec. In some cases, the first value is about 50 msec.

In some implementations, each of the temporal-relation values of the first feature vector is determined according to T=f(tᵢ−tⱼ, τ), in which f is any kernel function. Example kernel functions that can be used with the present equation can include exponential, linear, Gaussian, or polynomial function, but are not limited to these.

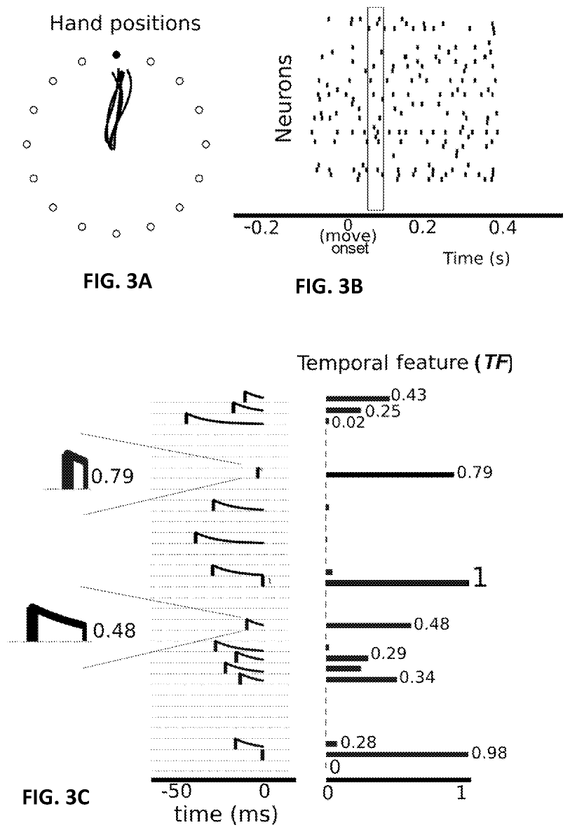

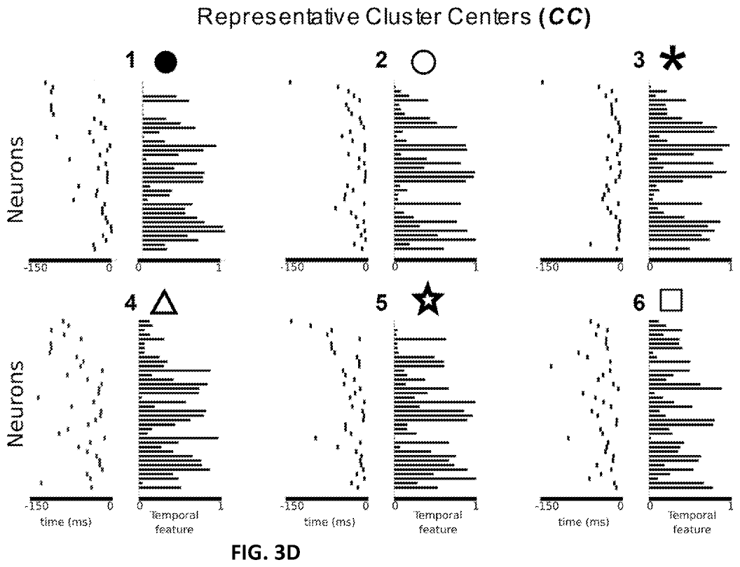

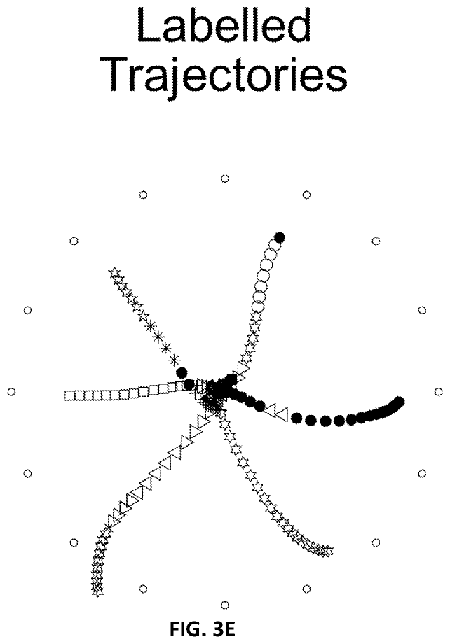

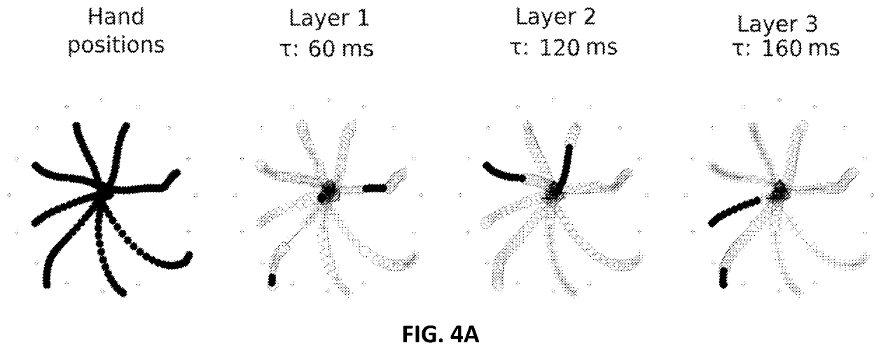

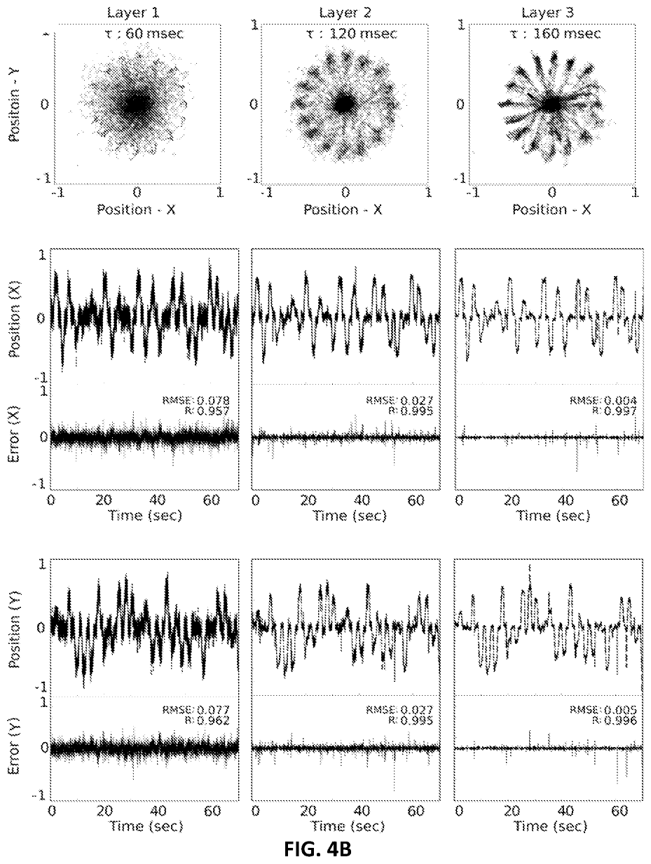

In one embodiment, the feature vector generator 220 determines a plurality of first layer feature vectors as described with respect to FIGS. 3A-3C. FIG. 3A shows repeated hand trajectories of a subject to the same target. FIG. 3B shows a plurality of spike trains recorded during one of the movements illustrated in FIG. 3A. Each dot/line segment represents a spike occurring at the neuron given by the y-axis and time given by the x-axis. Each row (e.g., horizontal row of dot/line segments) represents a sequence of spikes or action potentials from a different neuron. Action potentials from the neurons are recorded simultaneously and/or over time. The rectangle in FIG. 3B signifies or corresponds to a portion of the data displayed in FIG. 3C. As shown in FIG. 3C, one of the spikes is designated as a reference (neuron) spike (illustrated as corresponding long bar with value 1). Feature vectors are determined based on Equation 1, which are composed of values mapped using the relative delay between the reference neuron spike and spikes from all other neurons. The value of the decay at the instant the reference neuron spike occurs is used as an entry in the feature vector. Neuron spikes that occurred near in time to the reference spike have values closer to 1 as their delay are smaller while neuron spikes that occurred further in time to the reference spike have values closer to 0 as their delay are longer. Feature vectors may be calculated using Equation 1 for all sampled data, with each spike designated in turn as a reference spike. The plurality of feature vectors generated using Equation 1 for all sampled data can be subsequently grouped into the plurality of first layer feature vectors.

The first layer generator 230 includes or corresponds to a component that process, analyze, determines, or otherwise establishes a first layer comprising a first plurality of clusters, in one or more embodiments. The first layer can be analyzed, processed, generated, or determined from the plurality of first layer feature vectors.

An unsupervised clustering algorithm can be utilized to determine the first plurality of clusters. The unsupervised clustering algorithm can comprise a k-means clustering algorithm, a fuzzy k-means clustering algorithm, a hierarchical clustering, or a Gaussian mixture model (GMM), for example.

In some implementations, the unsupervised clustering algorithm comprises a k-means clustering algorithm. A K-means clustering algorithm is a distance-based clustering algorithm that partitions the data into a predetermined number of clusters. In some instances, an initial spatial location, or seed, is chosen and the clustering mechanism refines that seed location to the centroid through a repeated series of steps. The closer an initial seed is to the actual location of a centroid, the faster the clustering algorithm converges upon the centroid. In some instances, example open-source software for implementing k-mean clustering algorithm include, but are not limited to, Accord.NET, ALGLIB, AOSP, CrimeStat, ELKI, Julia, KNIME, Mahout, mlpack, Octave, OpenCV, Orange, PSPP, R, SciPy, Spark, Torch, and Weka.

In some instances, the unsupervised clustering algorithm comprises a fuzzy k-means clustering algorithm. Fuzzy clustering, also referred to as soft clustering or soft k-means, is a form of clustering in which the data point can belong to more than one cluster. Similar to k-means algorithms, a fuzzy k-means algorithm can also select an initial spatial location and the clustering mechanism refines the initial spatial location to the centroid through a repeated series of steps.

In some embodiments, the unsupervised clustering algorithm comprises a hierarchical clustering. Hierarchical clustering, also referred to as hierarchical cluster analysis or HCA, is a clustering analysis that build a hierarchy of clusters. In some instances, the hierarchical clustering utilizes an agglomerative strategy, or a bottom-up approach, in which each observation starts in its own cluster, and pairs of clusters are merged as one moves up the hierarchy. In other instances, the hierarchical clustering utilizes a divisive strategy, or a top-down strategy, in which all observations start in one cluster and splits are performed recursively as one moves down the hierarchy.

In some embodiments, the unsupervised clustering algorithm comprises a Gaussians mixture model (GMM). GMMs is a probabilistic model that assumes that there is a certain number of Gaussian distribution and each of the distribution represents a cluster.

In one embodiment, the first layer generator 230 establishes a first layer comprising a first plurality of clusters as described with respect to FIG. 3D. As illustrated in FIG. 3D, there are about 35 neurons. With each neuron referenced as a dimension, the 35-dimensional vectors can be sorted into about 50 clusters, in which six of the clusters are illustrated in FIG. 3D. The bar graphs can represent feature vectors located at the center of each cluster, or represent the point in the 35-dimensional space where the center of the cluster is located.

The cluster analyzer 240 includes or corresponds to a component that process, analyze, determines, establishes, or otherwise identifies a set of one or more clusters from the first plurality of clusters most similar to a cluster formed according to an incoming feature vector, in one or more embodiments. An incoming feature vector refers to a feature vector formed by an incoming spike train. In some instances, the incoming spike train is measured from the subject and is utilized to determine an activated cluster from a first plurality of clusters of the first layer feature vectors. An activated cluster refers to a set of one or more clusters from the first plurality of clusters of the first layer feature vectors that is similar or closest to the incoming feature vector (e.g., as compared to the rest of the first plurality of clusters).

The incoming feature vector comprises a set of temporal-relation values. Each temporal-relation value can be analyzed, calculated, or otherwise determined by any algorithms or equations that can process the temporal relationships from the plurality of spike times to fit a finite interval or range. For example, an increasing function or a decreasing function can process the temporal relationship from the plurality of spike times. The difference can be increased or decreased at a fixed or variable rate and/or interval. Each temporal-relation value from the set can further be determined according to Equation 2:

T=exp((−|tₙ−tⱼ|)/τ)

in which tₙ is a time instance of an additional reference spike train, tⱼ is a corresponding spike time of a neuron occurring immediately prior to the time instance of the additional reference spike train, τ is the first value, and T is the corresponding temporal-relation value, for instance. The additional reference spike train can be a spike train measured or recorded from the subject along with the incoming spike train to generate the set of temporal-relation values.

In one embodiment, the cluster analyzer 240 identifies the set of one or more clusters as described with respect to FIG. 3E. FIG. 3E illustrates six hand-motion trajectories. The different symbols along each trajectory indicate which of the clusters are “active” when the hand is at the spatial position that corresponds to the specific symbol. A cluster is “activated” when the incoming feature vector is closest to the cluster. For example, when a spike occurs, a feature vector is calculated and compared to all of the clusters. The cluster closest to the feature vector is termed or considered an activated cluster. FIG. 3E shows which cluster in FIG. 3D is activated at different points of the trajectories.

The second or successive layer generator 250 includes or corresponds to a component that processes, analyzes, establishes, or otherwise determines a second layer or a successive layer. The second or successive layer generator 250 can determine a plurality of second layer feature vectors comprising a second plurality of clusters that includes the set of one or more clusters from the first plurality of clusters. The second plurality of clusters can include a first cluster that comprises temporal-relation values of a cluster-subset of the second plurality of clusters. The temporal-relation values of the cluster-subset of the second plurality of clusters can be processed, analyzed, or otherwise determined from the set of one or more clusters and the incoming feature vector with respect to a time instance of a reference cluster of the cluster-sub set. The temporal-relation values can represent time-based, temporal relationships between clusters, obtained by translation/transformation of the temporal relationships to fit into a finite interval/range (e.g., a range of 0-1). The time instance may correspond to T having a second value. The second value can be larger than, smaller than, or equal to the first value. In some instances, the second value is larger than the first value.

The second value can be any value greater than 1 msec for instance, and can be up to 500000 msec for instance, according to various implementations. The second value can be from about 60 msec to about 150 msec. In some cases, the second value is from about 60 msec to about 145 msec, from about 60 msec to about 140 msec, from about 60 msec to about 135 msec, from about 60 msec to about 130 msec, from about 60 msec to about 125 msec, from about 60 msec to about 120 msec, from about 60 msec to about 115 msec, from about 60 msec to about 110 msec, from about 60 msec to about 105 msec, from about 60 msec to about 100 msec, from about 60 msec to about 95 msec, from about 60 msec to about 90 msec, from about 60 msec to about 80 msec, from about 70 msec to about 145 msec, from about 70 msec to about 140 msec, from about 70 msec to about 135 msec, from about 70 msec to about 130 msec, from about 70 msec to about 125 msec, from about 70 msec to about 120 msec, from about 70 msec to about 115 msec, from about 70 msec to about 110 msec, from about 70 msec to about 105 msec, from about 70 msec to about 100 msec, from about 70 msec to about 95 msec, from about 70 msec to about 90 msec, from about 80 msec to about 145 msec, from about 80 msec to about 140 msec, from about 80 msec to about 135 msec, from about 80 msec to about 130 msec, from about 80 msec to about 125 msec, from about 80 msec to about 120 msec, from about 80 msec to about 115 msec, from about 80 msec to about 110 msec, from about 80 msec to about 105 msec, from about 80 msec to about 100 msec, from about 80 msec to about 95 msec, from about 90 msec to about 145 msec, from about 90 msec to about 140 msec, from about 90 msec to about 135 msec, from about 90 msec to about 130 msec, from about 90 msec to about 125 msec, from about 90 msec to about 120 msec, from about 90 msec to about 115 msec, from about 90 msec to about 110 msec, from about 90 msec to about 105 msec, from about 90 msec to about 100 msec, from about 100 msec to about 145 msec, from about 100 msec to about 140 msec, from about 100 msec to about 135 msec, from about 100 msec to about 130 msec, from about 100 msec to about 125 msec, from about 100 msec to about 120 msec, from about 100 msec to about 115 msec, from about 100 msec to about 110 msec, from about 110 msec to about 145 msec, from about 110 msec to about 140 msec, from about 110 msec to about 135 msec, from about 110 msec to about 130 msec, from about 110 msec to about 125 msec, from about 110 msec to about 120 msec, from about 120 msec to about 145 msec, from about 120 msec to about 140 msec, from about 120 msec to about 135 msec, or from about 120 msec to about 130 msec. The second value is about 60 msec, 65 msec, 70 msec, 75 msec, 80 msec, 85 msec, 90 msec, 95 msec, 100 msec, 105 msec, 110 msec, 115 msec, 120 msec, 125 msec, 130 msec, 135 msec, 140 msec, 145 msec, or 150 msec. In some cases, the second value is about 100 msec.

Each of the temporal-relation values of the first cluster of the second plurality of clusters can be determined according to any decreasing function, such as according to T=f(tₘ−tⱼ, τ), in which f is any kernel function. Example kernel functions that can be used with the present equation can include exponential, linear, Gaussian, or polynomial function, but are not limited to these.

For instance, each of the temporal-relation values of the first cluster of the second plurality of clusters can be determined according to Equation 3:

T=exp((−|tₘ−tⱼ|)/τ)

in which tₘ is the time instance of the reference cluster, tⱼ is a corresponding spike time of a neuron occurring immediately prior to the time instance of the reference cluster, τ is the time constant having the second value, and T is the corresponding temporal-relation value, in an example embodiment. The reference cluster can be the activated cluster from a first plurality of clusters of the first layer feature vectors.

The second or successive layer generator 250 can establish a second layer comprising a first plurality of clusters of the second layer feature vectors. The second or successive layer generator 250 can further establish at least one successive layer after the second layer. Each of the at least one successive layer can comprise a successive set of clusters. Each of the at least one successive layer can further correspond to τ having a respective value. For each of the at least one successive layer, the τ of this layer can have a successively larger value than the second value. In some embodiments, τ for each of the at least one successive layer corresponds to a value of about 60 msec, about 70 msec, about 80 msec, about 90 msec, about 100 msec, about 110 msec, about 120 msec, about 130 msec, about 140 msec, about 150 msec, about 160 msec, about 170 msec, about 180 msec, about 190 msec, about 200 msec, or longer than 200 msec.

For example, to generate a third layer comprising a plurality of clusters, the second or successive layer generator 250 can identify another set of one or more clusters from the first plurality of clusters of the second layer feature vectors most similar to a cluster formed according to a second incoming feature vector. The second incoming feature vector can refer to a feature vector formed by an additional incoming spike train measured from the subject. A plurality of third layer feature vectors comprising a third plurality of clusters that includes the another set of one or more clusters can be determined based on the second incoming feature vector. The third plurality of clusters includes a first cluster that comprises temporal-relation values of a cluster-subset of the third plurality of clusters with respect to a time instance of a reference cluster of the cluster-subset of the third plurality of clusters. The time instance corresponds to τ having a third value (e.g., which can be different from other values associated with other layers). The second or successive layer generator 250 can establish a third layer comprising a first plurality of clusters of the third layer feature vectors.

Each of the temporal-relation values of the first cluster of the second plurality of clusters can be determined according to any decreasing function, such as according to T=f(tₒ−tⱼ, τ), in which f is any kernel function. Example kernel functions that can be used with the present equation can include exponential, linear, Gaussian, or polynomial function, but are not limited to these.

For example, each of the temporal-relation values of the first cluster of the third plurality of clusters can be determined according to Equation 4:

T=exp((−|tₒ−tⱼ|)/τ)

in which tₒ is the time instance of the reference cluster of the cluster-subset of the third plurality of clusters, tⱼ is a corresponding spike time of a neuron occurring immediately prior to the time instance of the reference cluster of the cluster-subset of the third plurality of clusters, τ is the time constant having the third value, and T is the corresponding temporal-relation value, in one example embodiment.

The third value can be any value greater than 1 msec for instance, and can be up to 500000 msec for instance, according to various implementations. The third value can be from about 110 msec to about 180 msec. In some cases, the third value is from about 110 msec to about 175 msec, from about 110 msec to about 170 msec, from about 110 msec to about 165 msec, from about 110 msec to about 160 msec, from about 110 msec to about 155 msec, from about 110 msec to about 150 msec, from about 110 msec to about 145 msec, from about 110 msec to about 140 msec, from about 110 msec to about 135 msec, from about 110 msec to about 130 msec, from about 120 msec to about 175 msec, from about 120 msec to about 170 msec, from about 120 msec to about 165 msec, from about 120 msec to about 160 msec, from about 120 msec to about 155 msec, from about 120 msec to about 150 msec, from about 120 msec to about 145 msec, from about 120 msec to about 140 msec, from about 130 msec to about 175 msec, from about 130 msec to about 170 msec, from about 130 msec to about 165 msec, from about 130 msec to about 160 msec, from about 130 msec to about 155 msec, from about 130 msec to about 150 msec, from about 130 msec to about 145 msec, from about 130 msec to about 140 msec, from about 150 msec to about 175 msec, from about 150 msec to about 170 msec, from about 150 msec to about 165 msec, from about 150 msec to about 160 msec, or from about 160 msec to about 175 msec. The third value can be about 110 msec, about 115 msec, about 120 msec, about 125 msec, about 130 msec, about 135 msec, about 140 msec, about 145 msec, about 150 msec, about 155 msec, about 160 msec, about 165 msec, about 170 msec, about 175 msec, or about 180 msec. In some cases, the third value is about 150 msec.

The second plurality of clusters and each successive set of clusters can each be independently determined by an unsupervised clustering algorithm. In some instances, the unsupervised clustering algorithm comprises a k-means clustering algorithm, a fuzzy k-means clustering algorithm, a hierarchical clustering, or a Gaussians mixture model (GMM). In some cases, the unsupervised clustering algorithm comprises a k-means clustering algorithm.

The motion correlator 260 includes or corresponds to a component that identifies, correlates, or otherwise determines a type of motion performed by the subject corresponds to one or more clusters of layer feature vectors of one of the at least one successive layer or the second layer, in one or more embodiments. A type of motion (sometimes simply referred as a “motion”) can include a motion/position/pose/action or a class/type/description thereof, can include continuous or incremental parts of a motion, or a temporal trail/path of motion, which can include one or a series/sequence/order of (related or independent) motions/positions/poses/actions intended and/or performed by a subject. The motion correlator 260 can determine a type of motion performed by the subject in relation to the one or more clusters of layer feature vectors using a supervised learning method. The supervised learning method can include a perceptron. Perceptron is an algorithm for supervised learning of binary classifiers. A binary classifier is a function which can decide whether or not an input, represented by a vector of numbers, belongs to some specific class. In some cases, each perceptron is a single-layer neural network that comprises an input value, a weight and bias, a net sum, and an activation function. A plurality of perceptrons (e.g., two, three, four, five, or more) can be utilized to determine whether the one or more clusters of layer feature vectors are consistent with a type of motion performed by the subject.

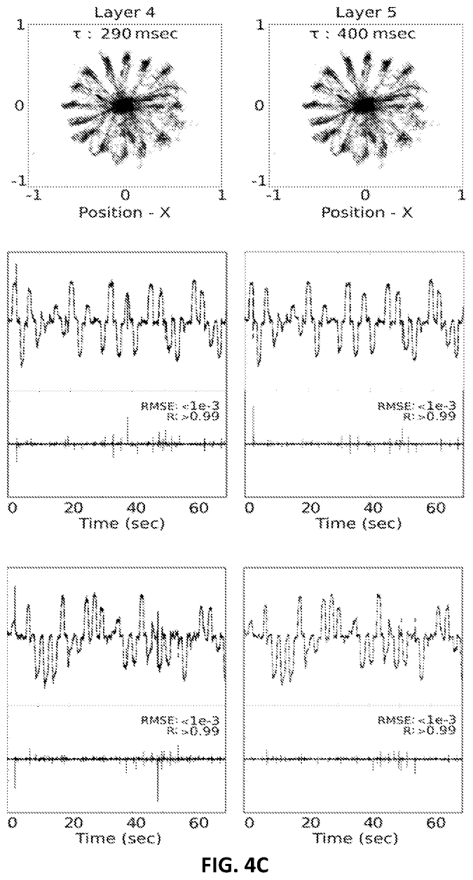

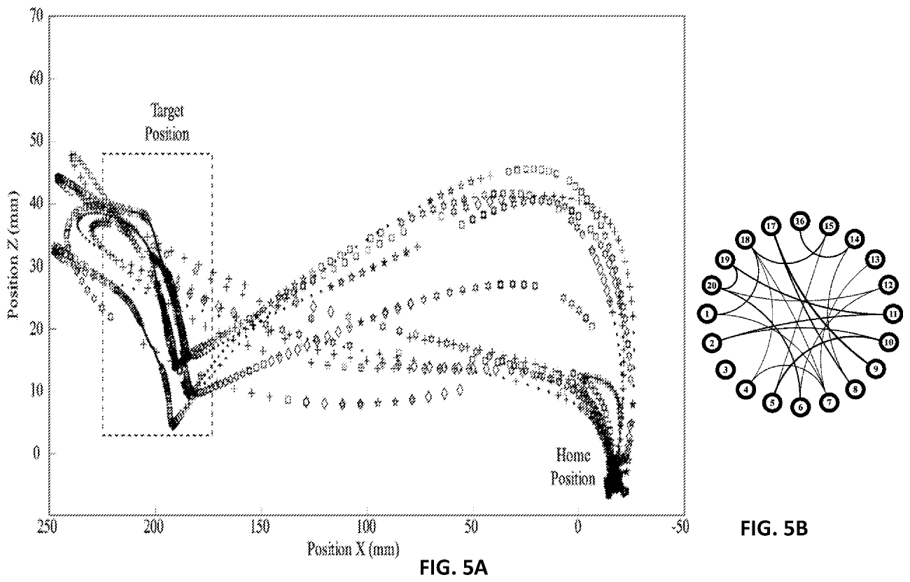

In one embodiment, the motion correlator 260 correlates a type of motion performed by a subject with one or more clusters of layer feature vectors as described with respect to FIGS. 4A, 4B and 4C. FIG. 4A shows the hand trajectories of the subject. First column of FIG. 4A shows the actual hand trajectories of the subject. Columns 2-4 show how the clusters that were activated at different points in the hand movement categories based on different layers. Each activated cluster is indicated by corresponding symbol and the symbols form a series of path positions indicating their respective movements by the subject. Layer 1 captures the different clusters that were activated sequentially as the hand moves toward a target. Layer 2 incorporates the sequence of activated clusters and further refines the activated clusters with respect to the respective hand movement. Layer 3 further optimizes the sequence of activated clusters, and yields the best performance out of the various layers in aligning with (or corresponding/correlating/mapping to, or representing/matching/predicting) the respective hand movement(s).

FIGS. 4B-4C show the correlation of the activated clusters mapped to the actual hand movement. Temporal features (or feature vectors) associated with each of the activated clusters of FIG. 4A are analyzed, processed, or otherwise decoded by a supervised learning method (e.g., by one or more perceptron) to locate or generate a unique pattern of the activated clusters comprising x- and y-positions that correlates with a specific hand movement trajectory. In the top panel under Layer 1, the actual hand movement trajectory is shown in black dot and the activated clusters are shown as unfilled ‘o’ dots. The second and third panel under Layer 1 shows the x and y positions separately. The black line in each of the second and third panel indicates the actual hand movement trajectory and the dashed lines indicate the predicted movements. In Layer 2 and Layer 3, the top panels show the actual hand movement trajectory in black dot and the respective activated clusters are shown as unfilled ‘o’ dots. The respective x and y positions are further shown as second and third panels under each layer. As shown in this example, Layer 3 provides the best performance out of the various layers, by providing distinct activated clusters indicating predicted hand movement trajectories that match closest to the actual hand movement trajectories when compared with the other layers. Further, Layer 3 indicates the lowest errors when compared with the other layers. Layers 4 and 5 show additional successive layers comprising additional clusters of successive layer feature vectors. No performance improvement is observed for Layers 4 and 5, indicating that the supervised learning method has best defined the correlation of the activated clusters with that of the actual hand movement trajectories. In some implementations or embodiments, the decay times for Layers 4 and/or 5 can be further adjusted/increased (e.g., to provide a larger parameter/cluster search space) to improve the matching/correlation performance of these layer(s) against actual hand movements. The improved matching/correlation performance may be observed to be similar or same as that for Layer 3, and this observation can be used to confirm/determine for instance that Layer 3 is already providing the best/optimal performance (since any improvement in performance in successive layers like Layer 4/5 would not significantly/meaningfully improve upon the performance provided by Layer 3).

The motion predictor 205 (comprising a feature vector generator 220, a first layer generator 230, a cluster analyzer 240, a second or successive layer generator 250, and/or a motion correlator 260) can further detect and determine an intended motion or implement the type of motion. The motion predictor 205 can detect a set of spike trains from the plurality of neurons, can determine that a first cluster based on the set of spike trains is consistent with the one or more clusters of layer feature vectors, and can determine that the first cluster is consistent with the one or more clusters of layer feature vectors. The motion predictor 205 can generate output data 270, which can indicate the subject's intended motion or for implementing the type of motion.

The motion predictor 205 can be configured to for instance wirelessly receive communication from an implanted device comprising one or more electrodes for detecting the plurality of spike trains. The implanted device can generate the plurality of spike trains 210. The implanted device can further update the motion predictor 205 with the plurality of spike trains 210. In one instance, the implanted device updates the motion predictor 205 continuously with the plurality of spike trains 210. In another instance, the implanted device updates the motion predictor 205 intermittently with the plurality of spike trains 210 (e.g., a portion of the spike trains recorded and stored up to each intermittent time instance). In an additional instance, the implanted device updates the motion predictor 205 at preset time intervals with the plurality of spike trains 210, for example, the preset time interval can be at about 10 minutes, 20 minutes, 30 minutes, 60 minutes, 2 hours, 3 hours, 4 hours, 5 hours, 6 hours, 12 hours, 24 hours, or more; or at intermittent time intervals. In a further instance, the implanted device updates the motion predictor 205 at the user's discretion.

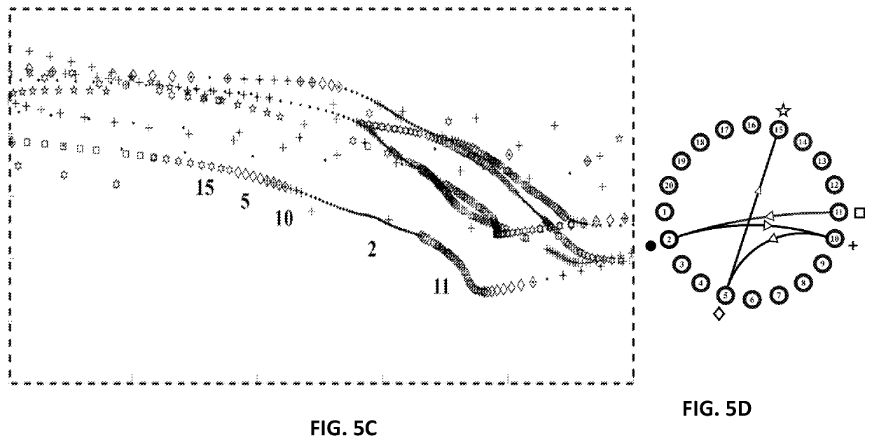

FIGS. 5A-5D show that temporal features (or feature vectors) can be predictable or stereotypical and show position related activation for a motion trajectory. For example, FIG. 5A shows the hand positions in a 2-dimentional graph and the corresponding clusters assigned to each position. The hand position moves between a home location and a target location. The corresponding clusters assigned to each position are plotted between the home location and the target location. The generation and clustering of temporal features are determined using an unsupervised clustering algorithm (e.g., a k-means clustering algorithm). The assignment of the clusters to the hand positions are done retrospectively. FIG. 5B shows that there is a causal connections between different clusters such that some clusters occur consistently after other clusters. FIG. 5C shows the zoomed positions from a target location from the dashed rectangle in FIG. 5A. In this location, a very specific sequence of cluster activation was found. This was established in the connectivity graph in FIG. 5D. The thickness of the line indicates the probability of interconnection between two activated clusters, with higher probability correlating to thicker lines.

### Methods of Processing Spike Trains

In certain embodiments, disclosed herein is a method utilizing the system 200 to determine a subject's intended motion. The method can utilize temporal dynamics to quantify and/or characterize spiking activity from a cerebral cortex. Intraspike intervals between different neurons can be characterized with exponential decays or other types of measures. Clustering can be used to find patterns of temporal dynamics using a specified time constant for the decays. Successive clustering with progressively longer time constants can be used to capture sequences of shorter-order temporal patterns. Patterns with a consistent relation to the subject's motion can be obtained and are further used for decoding motion information from neural activity. As used herein, an intraspike interval refers to a temporal-relation value between a neuron of interest and a reference neuron based on a time constant τ. In some cases, the temporal-relation value is calculated based on a spike time from the neuron of interest that occurred immediately prior to the time instance of the reference neuron (e.g., as a function of a duration between the spike time to the time instance). In some instances, the temporal-relation value is calculated according to any one of Equations 1-4.

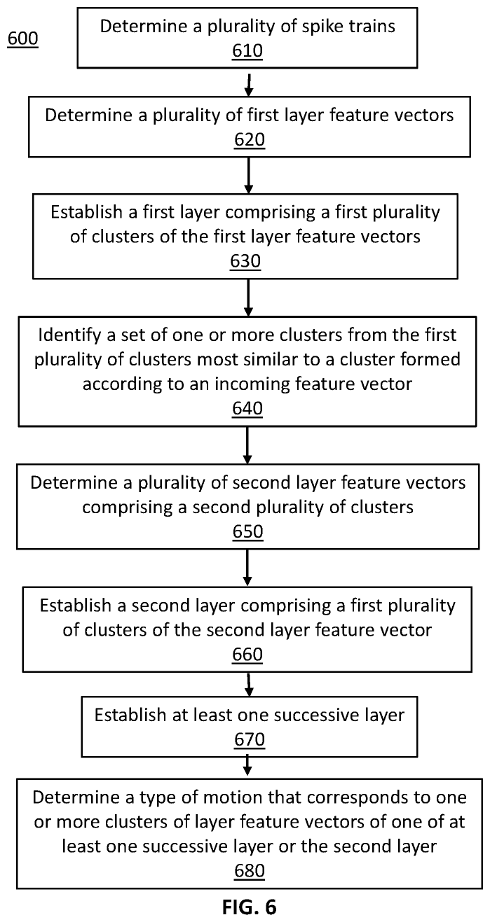

FIG. 6 is a flow chart illustrating a process of determining a type of motion that corresponds to one or more clusters of a layer, according to one or more disclosed embodiments. The process 600 is performed by the system 200. In some embodiments, the system 200 is implemented by a single system. In other embodiments, the system 200 is implemented by separate systems. In some embodiments, the process 600 includes more, fewer, or different steps than shown in FIG. 6.

In one approach, the motion predictor 205 obtains or determines a plurality of spike trains 610. A plurality of first layer feature vectors 620 are generated by or in conjunction with the feature vector generator 220. The first layer generator 230 further establishes a first layer comprising a first plurality of clusters of the first layer feature vector 630. A set of one or more clusters from the first plurality of clusters most similar to a cluster formed according to an incoming feature vector 640 are identified by the cluster analyzer 240. The second or successive layer generator 250 determines a plurality of second layer feature vectors comprising a second plurality of clusters 650. A second layer comprising a first plurality of clusters of the second layer feature vector 660 are established by the second or successive layer generator 250. The second or successive layer generator 250 can further establish at least one successive layer 670. A type of motion that corresponds to one or more clusters of layer feature vectors of one of at least one successive layer or the second layer 680 is determined by the motion correlator 260.

In further detail of 610, and in some embodiments, the feature vector generator 220 (comprising at least one processor) determines a plurality of spike trains. The feature vector generator 220 may be activated based on availability or occurrence of neural activity, e.g., availability or occurrence of one or more spike trains. The feature vector generator 220 may receive, measure, detect, access, or otherwise determine the plurality of spike trains (or information about the spike trains), corresponding to a plurality/population of neurons of a cerebral cortex of the mammal or subject. For instance, the feature vector generator 220 may receive or access information about the plurality of spike trains from a measurement/detection module implanted or attached to the mammal or subject. In some embodiments, the feature vector generator 220 may receive or access the information from a memory or other storage device. The feature vector generator 220 may receive or access the information in real time, or in batch at various time instances (e.g., after the information is collected and accumulated over a corresponding time duration). The feature vector generator 220 may receive the information which includes, indicates, or tracks time instance(s) at which spike(s) are fired from corresponding identified neurons. The feature vector generator 220 may determine the plurality of spike trains for use in determining at least one feature vector.

In further detail of 620, and in some embodiments, the feature vector generator 220 may determine a plurality of first layer feature vectors. A plurality of first layer feature vectors can be generated by or determined by the feature vector generator 220. The feature vector generator 220 may generate or determine the plurality of first layer feature vectors 620 using one or more algorithms or equations based on user input or based on a predetermined program. The one or more algorithms or equations can be any algorithms or equations that can process the temporal relationships from the plurality of spike times to fit a finite interval or range. The feature vector generator 220 may use an algorithms or equation to generate or determine a feature vector characterized with exponential decay, optionally can use Equation 1 to generate or determine a feature vector characterized with exponential decay. The feature vector generator 220 may also use an algorithms or equation to generate or determine a feature vector characterized with exponential growth. If Equation 1 is selected, the feature vector generator 220 may select a reference spike train randomly, based on user input, or based on a predetermined program. The feature vector generator 220 may further select a first value randomly from about 5 msec to about 80 msec, select the first value based on user input, and/or via a parameter search (e.g., selecting a number of candidate values, and performing training, decoding or cluster identification, and error/similarity evaluation against a cluster formed according to an incoming feature vector, as described herein, for each of the candidate values, so as to identify one of the candidate values that provides a best/optimal/suitable/acceptable performance according to the error/similarity evaluation), or select a preprogramed value as the first value.

In further detail of 630, and in some embodiments, the feature vector generator 220 may establish a first layer comprising a plurality of clusters of the first layer feature vectors. The first layer generator 230 (comprising at least one processor) may generate or determine a first layer comprising a first plurality of clusters of the first layer feature vector. The first layer generator 230 may analyze, process, generate, or determine the first layer from the plurality of first layer feature vectors using an unsupervised clustering algorithm, e.g., a k-means clustering algorithm, a fuzzy k-means clustering algorithm, a hierarchical clustering, or a Gaussian mixture model (GMM). The first layer generator 230 may select the unsupervised clustering algorithm based on user input or based on a predetermined program. In one embodiment, the first layer generator 230 selects a k-means clustering algorithm to analyze, process, generate, or determine the first layer from the plurality of first layer feature vectors.

In further detail of 640, and in some embodiments, a cluster analyzer 240 may identify a set of one or more clusters from the first plurality of clusters most similar to a cluster formed according to an incoming feature vector. The cluster analyzer 240 (comprising at least one processor) may generate or determine a set of one or more clusters from the first plurality of clusters most similar to a cluster formed according to an incoming feature vector. The feature vector generator 220 may receive, measure, detect, access, or otherwise determine spike trains used to determine the incoming feature vector. The spike trains for use in determining the incoming feature vector may be determined immediately after determining the plurality of spike trains 610. Alternatively, the spike trains for use in determining the incoming feature vector may be determined after a set time after determining the plurality of spike trains 610, for example, after about 10 minutes, 20 minutes, 30 minutes, 60 minutes, 2 hours, 4 hours, 6 hours, 12 hours, 24 hours, 48 hours, 72 hours, or longer. The spike trains for use in determining the incoming feature may be selected by the feature vector generator 220 based on user input, may be selected randomly, or may be selected based on a predetermined program. The feature vector generator 220 may analyze, calculate, or otherwise determine a set of temporal-relation values encompassed by the incoming feature and transfer the set to the cluster analyzer 240. The feature vector generator 220 may use an algorithm or equation to generate or determine the set of temporal-relation values characterized with exponential decays, for example, may use Equation 2. Alternatively, the feature vector generator 220 may use an algorithm or equation to generate or determine the set of temporal-relation values characterized with exponential growths. The selection of the algorithm or equation may be based on user input or based on a predetermined program.

In further detail of 650, and in some embodiments, a second or successive layer generator 250 may determine a plurality of second layer feature vectors comprising a second plurality of clusters. The second or successive layer generator 250 (comprising at least one processor) may generate or determine a plurality of second layer feature vectors comprising a second plurality of clusters. The second plurality of clusters may include a first cluster comprising temporal-relation values that can be processed, analyzed, or otherwise determined from the set of one or more clusters determined by the cluster analyzer 240 and the incoming feature vector determined by the feature vector generator 220. The second or successive layer generator 250 may use an algorithm or equation to generate or determine the first cluster comprising temporal-relation values characterized with exponential decay, for example, may use Equation 3. The second or successive layer generator 250 may select the second value randomly, may select the second value based on user input, or may select a predetermined value as the second value (e.g., which can be different from other values associated with other layers). The second or successive layer generator 250 may also use an algorithm or equation to generate or determine the first cluster comprising temporal-relation values characterized with exponential growth.

In further details of 660 and 670, and in some embodiments, the second or successive layer generator 250 may establish a second layer comprising a first plurality of clusters of the second layer feature vector and optionally, at least one successive layer. The second or successive layer generator 250 may select a value for T for use in determining the second layer 660 or the at least one successive layer 670 randomly, based on use input, or select a predetermined value. If a successive layer 670 is determined, the value T for the at least one successive layer 670 selected by the second or successive layer generator 250 is larger than the value T for the second layer 660. The at least one successive layer 670 can be a third layer, fourth layer, fifth layer, sixth layer, seventh layer, eighth layer, ninth layer, tenth layer, or more.

In one embodiment, the second or successive layer generator 250 may establish a third layer. The second or successive layer generator 250 may use an algorithm or equation to generate or determine a plurality of third layer feature vectors comprising temporal-relation values characterized with exponential decay, for example, may use Equation 4. The second or successive layer generator 250 may select the third value randomly, may select the third value based on user input, or may select a predetermined value as the third value. The second or successive layer generator 250 may also use an algorithm or equation to generate or determine the plurality of third layer feature vectors comprising temporal-relation values characterized with exponential growth.

In further detail of 680, and in some embodiments, a motion correlator 260 may determine a type of motion that corresponds to one or more clusters of layer feature vectors of one of at least one successive layer or the second layer. In some embodiments, a type of motion that corresponds to one or more clusters of layer feature vectors of one of at least one successive layer or the second layer is generated by or determined by the motion correlator 260. The motion correlator 260 can determine the type of motion performed by the subject in relation to the one or more clusters of layer feature vector using a supervised learning method, e.g., using perceptron. The motion correlator 260 may select a supervised learning method, e.g., perceptron, by a predetermined program or by user input. If perceptron is selected, the motion correlator 260 may use a plurality of perceptrons, e.g., two, three, four, five, or more to determine whether the one or more clusters of layer feature vectors are consistent with a type of motion performed by the subject.

The motion correlator 260 may process one or more clusters of the successive layer 670 generated by the second or successive layer generator 250 and correlate the one or more clusters to a type of motion. The second or successive layer generator 250 may establish one, two, three, four, five, six, seven, eight, nine, ten or more successive layers. The motion correlator 260 may process one, two, three, four, five, six, seven, eight, nine, ten, or all of the successive layers generated by the second or successive layer generator 250. The motion correlator 260 may process the successive layers until an error threshold is reached, e.g., an error value of about 5%, 10%, 15%, or 20% is reached. In one instance, the error threshold is defined based on user input. In another instance, the error threshold is defined based on a predetermined value. In an additional instance, the error threshold is adjusted in situ based on the correlation with the type of motion. For example, the motion correlator 260 may calculate the error value as a mean squared difference between the recorded positions and the predicted positions from a motion predictor. Multiple (successive) layers can be established, each with its own motion predictor. The best motion predictor can be chosen as the one that gives the minimum prediction error. In some implementations, the error value is calculated or determined as a mean squared error (MSE) or as a mean squared deviation (MSD). The motion correlator 260 may process one or more clusters of the successive layer 670 and generate an error value (e.g., MSE or MSD) with the first layer and with each successive layer. The motion correlator 260 may further compare the error values from the first and each successive layers to select the layer with the lowest or minimum error value. A successive layer can be over-refined if it is determined subsequently to the layer with the minimum error value and the associated error value of the successive layer is higher than the minimum error value. For example, a fourth layer may be over-refined if its associated error value is higher than the error value of the third layer and the third layer provides the minimum error value.

In one approach, the process 600 is performed on a mammal. The mammal can be a non-human primate and the process 600 can be performed on the non-human primate. The mammal can also be a human and the process 600 can be performed on the human. The mammal can further be a non-primate and the process 600 can be performed on the non-primate.

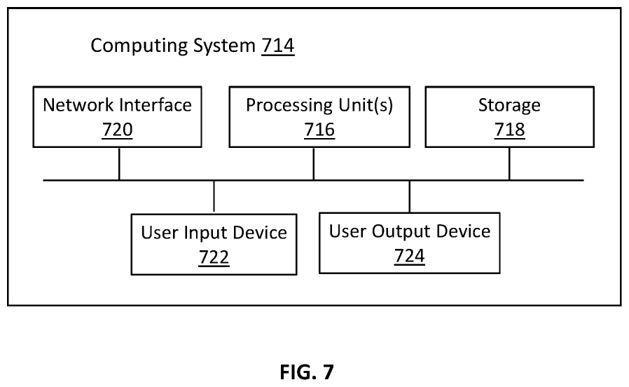

Various operations described herein can be implemented on one or more computer systems. FIG. 7 shows a block diagram of a representative computing system 714 usable to implement the present disclosure. In some embodiments, the system 200 is implemented by the computing system 714. Computing system 714 can be implemented, for example, as a consumer device such as a smartphone, other mobile phone, tablet computer, wearable computing device (e.g., smart watch, eyeglasses, head mounted display), desktop computer, laptop computer, cloud computing service or implemented with distributed computing devices. In some embodiments, the computing system 714 can include computer components such as processors 716, storage device 718, network interface 720, user input device 722, and user output device 724.

Network interface 720 can provide a connection to a wide area network (e.g., the Internet) to which WAN interface of a remote server system is also connected. Network interface 720 can include a wired interface (e.g., Ethernet) and/or a wireless interface implementing various RF data communication standards such as Wi-Fi, Bluetooth, or cellular data network standards (e.g., 3G, 4G, 5G, 60 GHz, LTE, etc.).

User input device 722 can include any device (or devices) via which a user can provide signals to computing system 714; computing system 714 can interpret the signals as indicative of particular user requests or information. User input device 722 can include any or all of a keyboard, touch pad, touch screen, mouse or other pointing device, scroll wheel, click wheel, dial, button, switch, keypad, microphone, sensors (e.g., a motion sensor, an eye tracking sensor, etc.), and so on.

User output device 724 can include any device via which computing system 714 can provide information to a user. For example, user output device 724 can include a display to show images generated by or delivered to computing system 714. The display can incorporate various image generation technologies, e.g., a liquid crystal display (LCD), light-emitting diode (LED) including organic light-emitting diodes (OLED), projection system, cathode ray tube (CRT), or the like, together with supporting electronics (e.g., digital-to-analog or analog-to-digital converters, signal processors, or the like). A device such as a touchscreen that function as both input and output device can be used. Output devices 724 can be provided in addition to or instead of a display. Examples include indicator lights, speakers, tactile “display” devices, printers, and so on.

Some implementations include electronic components, such as microprocessors, storage and memory that store computer program instructions in a non-transitory computer readable storage medium. Many of the features described in this specification can be implemented as processes that are specified as a set of program instructions encoded on a computer readable storage medium. When these program instructions are executed by one or more processors, they cause the processors to perform various operation indicated in the program instructions. Examples of program instructions or computer code include machine code, such as is produced by a compiler, and files including higher-level code that are executed by a computer, an electronic component, or a microprocessor using an interpreter. Through suitable programming, processor 716 can provide various functionality for computing system 714, including any of the functionality described herein as being performed by a server or client, or other functionality associated with message management services.

It will be appreciated that computing system 714 is illustrative and that variations and modifications are possible. Computer systems used in connection with the present disclosure can have other capabilities not specifically described here. Further, while computing system 714 is described with reference to particular blocks, it is to be understood that these blocks are defined for convenience of description and are not intended to imply a particular physical arrangement of component parts. For instance, different blocks can be located in the same facility, in the same server rack, or on the same motherboard. Further, the blocks need not correspond to physically distinct components. Blocks can be configured to perform various operations, e.g., by programming a processor or providing appropriate control circuitry, and various blocks might or might not be reconfigurable depending on how the initial configuration is obtained. Implementations of the present disclosure can be realized in a variety of apparatus including electronic devices implemented using any combination of circuitry and software.

Having now described some illustrative implementations, it is apparent that the foregoing is illustrative and not limiting, having been presented by way of example. In particular, although many of the examples presented herein involve specific combinations of method acts or system elements, those acts and those elements can be combined in other ways to accomplish the same objectives. Acts, elements and features discussed in connection with one implementation are not intended to be excluded from a similar role in other implementations or implementations.

The hardware and data processing components used to implement the various processes, operations, illustrative logics, logical blocks, modules and circuits described in connection with the embodiments disclosed herein may be implemented or performed with a general purpose single- or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device, etc.) may include one or more devices (e.g., RAM, ROM, Flash memory, hard disk storage, etc.) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an embodiment, the memory is communicably connected to the processor via a processing circuit and includes computer code for executing (e.g., by the processing circuit and/or the processor) the one or more processes described herein.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM (e.g., at least one resistive RAM (ReRAM or RRAM) or memristor), ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular can also embrace implementations including a plurality of these elements, and any references in plural to any implementation or element or act herein can also embrace implementations including only a single element. References in the singular or plural form are not intended to limit the presently disclosed systems or methods, their components, acts, or elements to single or plural configurations. References to any act or element being based on any information, act or element can include implementations where the act or element is based at least in part on any information, act, or element.

Any implementation disclosed herein can be combined with any other implementation or embodiment, and references to “an implementation,” “some implementations,” “one implementation” or the like are not necessarily mutually exclusive and are intended to indicate that a particular feature, structure, or characteristic described in connection with the implementation can be included in at least one implementation or embodiment. Such terms as used herein are not necessarily all referring to the same implementation. Any implementation can be combined with any other implementation, inclusively or exclusively, in any manner consistent with the aspects and implementations disclosed herein.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements. Technical and scientific terms used herein have the meanings commonly understood by one of ordinary skill in the art, unless otherwise defined. Any suitable materials and/or methodologies known to those of ordinary skill in the art can be utilized in carrying out the methods described herein.

Systems and methods described herein may be embodied in other specific forms without departing from the characteristics thereof. As used herein, “approximately,” “about” “substantially” or other terms of degree will be understood by persons of ordinary skill in the art and will vary to some extent on the context in which it is used. If there are uses of the term which are not clear to persons of ordinary skill in the art given the context in which it is used, references to “approximately,” “about” “substantially” or other terms of degree shall include variations of +/−10% from the given measurement, unit, or range unless explicitly indicated otherwise.

Coupled elements can be electrically, mechanically, or physically coupled with one another directly or with intervening elements. Scope of the systems and methods described herein is thus indicated by the appended claims, rather than the foregoing description, and changes that come within the meaning and range of equivalency of the claims are embraced therein.

The term “coupled” and variations thereof includes the joining of two members directly or indirectly to one another. Such joining may be stationary (e.g., permanent or fixed) or moveable (e.g., removable or releasable). Such joining may be achieved with the two members coupled directly with or to each other, with the two members coupled with each other using a separate intervening member and any additional intermediate members coupled with one another, or with the two members coupled with each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References to “or” can be construed as inclusive so that any terms described using “or” can indicate any of a single, more than one, and all of the described terms. A reference to “at least one of ‘A’ and ‘B’” can include only ‘A’, only ‘B’, as well as both ‘A’ and ‘B’. Such references used in conjunction with “comprising” or other open terminology can include additional items.

Modifications of described elements and acts such as variations in sizes, dimensions, structures, shapes and proportions of the various elements, values of parameters, mounting arrangements, use of materials, colors, orientations can occur without materially departing from the teachings and advantages of the subject matter disclosed herein. For example, elements shown as integrally formed can be constructed of multiple parts or elements, the position of elements can be reversed or otherwise varied, and the nature or number of discrete elements or positions can be altered or varied. Other substitutions, modifications, changes and omissions can also be made in the design, operating conditions and arrangement of the disclosed elements and operations without departing from the scope of the present disclosure.

References herein to the positions of elements (e.g., “top,” “bottom,” “above,” “below”) are merely used to describe the orientation of various elements in the FIGURES. The orientation of various elements may differ according to other exemplary embodiments, and that such variations are intended to be encompassed by the present disclosure.

As used herein, a subject can be a mammal, such as a non-primate (e.g., cows, pigs, horses, cats, dogs, rats, etc.) or a primate (e.g., monkey and human). In certain embodiments, the term “subject,” as used herein, refers to a vertebrate, such as a mammal. Mammals include, without limitation, humans, non-human primates, wild animals, feral animals, farm animals, sport animals, and pets. In certain exemplary embodiments, a subject is a human.

As used herein, the terms “subject” and “user” are used interchangeably.

Unless otherwise defined, all technical and scientific terms used herein have the same meaning as commonly understood by one of ordinary skill in the art to which this disclosure belongs. Although methods and materials similar or equivalent to those described herein can be used in the practice or testing of the present disclosure, suitable methods and materials are described herein.

As used herein, the singular forms “a”, “an,” and “the” include plural referents unless the context clearly indicates otherwise. For example, the term “a cell” includes a plurality of cells, including mixtures thereof.

As used herein, the term “about” is used to indicate that a value includes the standard deviation of error for the device or method being employed to determine the value. The term “about” when used before a numerical designation, e.g., temperature, time, amount, and concentration, including range, indicates approximations which may vary by (+) or (−) 15%, 10%, 5%, 3%, 2%, or 1%.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

### EXAMPLES

These examples are provided for illustrative purposes only and not to limit the scope of the claims provided herein.

### Example 1—Spike Pattern Interactions in Macaque Motor Cortex

Two adult male monkeys (Macaca mulatta, R and N) were used in a study. All procedures for the care and use of these animals were in accordance with the guidelines of the NIH and were approved by the Institutional Animal Care and Use Committee of the University of Pittsburgh.

Behavioral Task

Monkeys were trained to perform reaches with their right arms while viewing a virtual reality (VR) environment through a depth-displaying monitor (Virtual Window, Dimension Technologies Inc.). The monkeys could not see their own arms during the task; instead, they were shown a spherical cursor in VR representing their arm positions. Cursor radii were about 0.3 cm (all such measures refer to length in physical space). Hand positions were tracked optically at 50 Hz using an Optotrak 3020 motion capture system (Northern Digital Inc.). The monkeys performed several variations of the center-out reaching task. In each task, monkeys began a trial by holding their arms steady for 500-700 ms in the center of the workspace volume, marked by a spherical target with a radius of 0.6 cm. A peripheral target of the same size then appeared, cueing the monkey to begin moving. Targets could appear at one of 16 evenly spaced positions around the home position, forming a circle with a 6 cm radius. The targets were arranged in the vertical plane, though the task required 3D movement control; reaches in front of or behind a target were not rewarded. Monkeys were permitted 600-800 ms to reach and hold position in the target and received a liquid reward for each successful trial. Each session was split into two blocks with a roughly equal number of trials. The processing of the scene displayed in VR during a reach may be another factor driving changes in M1 firing patterns. That is, visual feedback from the cursor movement may drive firing. To dissociate these processes, the study randomly selected 50% of the trials and disabled the cursor display before the movement began, 140 ms after the target appeared (“invisible trials”). The monkeys were not cued commonly require stopping and holding within a target zone, which is difficult without visual feedback. The study therefore omitted the terminal hold period for tasks used in this study; monkeys were rewarded upon touching the target with the cursor.

Neuronal Recordings

Monkeys were chronically implanted with 96-channel microelectrode “Utah” arrays (Blackrock Microsystems), which were inserted into the arm area of the primary motor cortex. Monkey R was implanted with two arrays but data from only one array was used in this study. Extracellular voltage signals measured from the electrode arrays were amplified, filtered, and digitized using either a Plexon MAP system (Plexon Inc.) or a Blackrock Cerebus system (Blackrock Microsystems). Spike waveforms were sorted manually offline using Plexon Offline Sorter (OFS, Plexon Inc.). Unit isolation was judged based on waveform, cluster separation, inter-spike interval histogram, autocorrelation, and cross-correlation with other units on the same electrode. Only well-isolated units were saved for further analysis.

While certain embodiments have been illustrated and described, it should be understood that changes and modifications can be made therein in accordance with ordinary skill in the art without departing from the technology in its broader aspects as defined in the following claims.

The embodiments, illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the claimed technology. Additionally, the phrase “consisting essentially of” will be understood to include those elements specifically recited and those additional elements that do not materially affect the basic and novel characteristics of the claimed technology. The phrase “consisting of” excludes any element not specified.

The present disclosure is not to be limited in terms of the particular embodiments described in this application. Many modifications and variations can be made without departing from its spirit and scope, as will be apparent to those skilled in the art. Functionally equivalent methods and compositions within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, which can of course vary. It is also to be understood that the terminology used herein is for the purpose of describing particular embodiments only, and is not intended to be limiting.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, particularly in terms of providing a written description, all ranges disclosed herein also encompass any and all possible subranges and combinations of subranges thereof, inclusive of the endpoints. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like, include the number recited and refer to ranges which can be subsequently broken down into subranges as discussed above. Finally, as will be understood by one skilled in the art, a range includes each individual member.

All publications, patent applications, issued patents, and other documents referred to in this specification are herein incorporated by reference as if each individual publication, patent application, issued patent, or other document was specifically and individually indicated to be incorporated by reference in its entirety. Definitions that are contained in text incorporated by reference are excluded to the extent that they contradict definitions in this disclosure.

Other embodiments are set forth in the following claims.

1. A method comprising:
determining, by at least one processor, a plurality of spike trains from a plurality of neurons of a cerebral cortex of a subject;
determining, by the at least one processor, a plurality of first layer feature vectors, the plurality of first layer feature vectors including a first feature vector that comprises temporal-relation values of a subset of the plurality of spike trains with respect to a time instance of a reference spike train of the subset, corresponding to a time constant τ having a first value;
establishing, by the at least one processor, a first layer comprising a first plurality of clusters of the first layer feature vectors;
identifying, by the at least one processor, a set of one or more clusters from the first plurality of clusters most similar to a cluster formed according to an incoming feature vector;
determining, by the at least one processor, a plurality of second layer feature vectors comprising a second plurality of clusters that includes the set of one or more clusters, the second plurality of clusters including a first cluster that comprises temporal-relation values of a cluster-subset of the second plurality of clusters with respect to a time instance of a reference cluster of the cluster-subset, corresponding to τ having a second value;
establishing, by the at least one processor, a second layer comprising a first plurality of clusters of the second layer feature vectors;
establishing, by the at least one processor, at least one successive layer after the second layer, each of the at least one successive layer corresponding to τ having a respective value; and
determining, by the at least one processor, that a type of motion performed by the subject corresponds to one or more clusters of layer feature vectors of one of the at least one successive layer or the second layer.

2. The method of claim 1, comprising:
detecting, by the at least one processor, a set of spike trains from the plurality of neurons;
determining, by the at least one processor, that a first cluster based on the set of spike trains is consistent with the one or more clusters of layer feature vectors; and
determining, by the at least one processor responsive to determining that the first cluster is consistent with the one or more clusters of layer feature vectors, that the subject is intending or implementing the type of motion.

3. (canceled)

4. The method of claim 1, wherein the second value is larger than the first values, and each of the at least one successive layer corresponds to T having a successively larger value than the second value.

5. The method of claim 1, wherein each of the temporal-relation values of the first feature vector is indicative of a difference in corresponding spike time with respect to the time instance of the reference spike train of the subset.

6. The method of claim 1, comprising determining, by the at least one processor, each of the temporal-relation values of the first feature vector according to:
T=exp((−|tᵢ−tⱼ|)/τ),
wherein:
tᵢ is the time instance of the reference spike train,
tⱼ is a corresponding spike time of a neuron occurring immediately prior to the time instance of the reference spike train,
τ is the time constant having the first value, and
T is the corresponding temporal-relation value.

7.-8. (canceled)

9. The method of claim 1, wherein each of the temporal-relation values of the first cluster is indicative of a difference in corresponding spike time with respect to the time instance of the reference spike train of the cluster-subset.

10. The method of claim 1, comprising determining, by the at least one processor, each of the temporal-relation values of the first cluster of the second plurality of clusters according to:
T=exp((−|tₘ−tⱼ|)/τ),
wherein:
tₘ is the time instance of the reference cluster,
tⱼ is a corresponding spike time of a neuron occurring immediately prior to the time instance of the reference cluster,
τ is the time constant having the second value, and
T is the corresponding temporal-relation value.

11.-12. (canceled)

13. The method of claim 1, wherein the first plurality of clusters and the second plurality of clusters are each independently determined by an unsupervised clustering algorithm.

14. The method of claim 13, wherein the unsupervised clustering algorithm comprises a k-means clustering algorithm, a fuzzy k-means clustering algorithm, a hierarchical clustering, or a Gaussians mixture model (GMM).

15. The method of claim 1, wherein the incoming feature vector comprises a set of temporal-relation values, wherein each temporal-relation value from the set is determined according to:
T=exp((−|tₙ−tⱼ|)/τ),
wherein:
tₙ is a time instance of an additional reference spike train,
tⱼ is a corresponding spike time of a neuron occurring immediately prior to the time instance of the additional reference spike train,
τ is the first value, and
T is the corresponding temporal-relation value.

16. (canceled)

17. The method of claim 1, further comprising:
identifying, by the at least one processor, another set of one or more clusters from the first plurality of clusters of the second layer feature vectors most similar to a cluster formed according to a second incoming feature vector,
determining, by the at least one processor, a plurality of third layer feature vectors comprising a third plurality of clusters that includes the another set of one or more clusters, the third plurality of clusters including a first cluster that comprises temporal-relation values of a cluster-subset of the third plurality of clusters with respect to a time instance of a reference cluster of the cluster-subset of the third plurality of clusters, corresponding to τ having a third value; and
establishing, by the at least one processor, a third layer comprising a first plurality of clusters of the third layer feature vectors.

18. The method of claim 17, comprising determining, by the at least one processor, each of the temporal-relation values of the first cluster of the third plurality of clusters according to:
T=exp((−|tₒ−tⱼ|)/τ),
wherein:
tₒ is the time instance of the reference cluster of the cluster-subset of the third plurality of clusters,
tⱼ is a corresponding spike time of a neuron occurring immediately prior to the time instance of the reference cluster of the cluster-subset of the third plurality of clusters,
τ is the time constant having the third value, and
T is the corresponding temporal-relation value.

19.-20. (canceled)

21. The method of claim 2, wherein the first cluster is determined to be consistent with the one or more clusters of layer feature vectors using a supervised learning method.

22. The method of claim 21, wherein the supervised learning method comprises using a perceptron.

23. (canceled)

24. A system comprising:
at least one processor configured to:
determine a plurality of spike trains from a plurality of neurons of a cerebral cortex of a subject;
determine a plurality of first layer feature vectors, the plurality of first layer feature vectors including a first feature vector that comprises temporal-relation values of a subset of the plurality of spike trains with respect to a time instance of a reference spike train of the subset, corresponding to a time constant τ having a first value;
establish a first layer comprising a first plurality of clusters of the first layer feature vectors;
identify a set of one or more clusters from the first plurality of clusters most similar to a cluster formed according to an incoming feature vector;
determine a plurality of second layer feature vectors comprising a second plurality of clusters that includes the set of one or more clusters, the second plurality of clusters including a first cluster that comprises temporal-relation values of a cluster-subset of the second plurality of clusters with respect to a time instance of a reference cluster of the cluster-subset, corresponding to τ having a second value;
establish a second layer comprising a first plurality of clusters of the second layer feature vectors;
establish at least one successive layer after the second layer, each of the at least one successive layer corresponding to τ having a respective value; and
determine that a type of motion performed by the subject corresponds to one or more clusters of layer feature vectors of one of the at least one successive layer or the second layer.

25. The system of claim 24, wherein the at least one processor is configured to:
detect a set of spike trains from the plurality neurons;
determine a first cluster based on the set of spike trains is consistent with the one or more clusters of layer feature vectors; and
determine whether the subject is intending or implementing the type of motion by determining that the first cluster is consistent with the one or more clusters of layer feature vectors.

26.-37. (canceled)

38. The system of claim 24, wherein the at least one processor is further configured to:
identify another set of one or more clusters from the first plurality of clusters of the second layer feature vectors most similar to a cluster formed according to a second incoming feature vector,
determine a plurality of third layer feature vectors comprising a third plurality of clusters that includes the another set of one or more clusters, the third plurality of clusters including a first cluster that comprises temporal-relation values of a cluster-subset of the third plurality of clusters with respect to a time instance of a reference cluster of the cluster-subset of the third plurality of clusters, corresponding to τ having a third value; and
establish a third layer comprising a first plurality of clusters of the third layer feature vectors.

39.-40. (canceled)

41. The system of claim 24, wherein the at least one processor is configured to wirelessly receive communication from an implanted device comprising one or more electrodes for detecting the plurality of spike trains.

42. (canceled)

43. A non-transitory computer-readable medium storing a program including instructions that, when executed by a processor,
determines a plurality of spike trains from a plurality of neurons of a cerebral cortex of a subject;
determines a plurality of first layer feature vectors, the plurality of first layer feature vectors including a first feature vector that comprises temporal-relation values of a subset of the plurality of spike trains with respect to a time instance of a reference spike train of the subset, corresponding to a time constant τ having a first value;
establishes a first layer comprising a first plurality of clusters of the first layer feature vectors;
identifies a set of one or more clusters from the first plurality of clusters most similar to a cluster formed according to an incoming feature vector;
determines a plurality of second layer feature vectors comprising a second plurality of clusters that includes the set of one or more clusters, the second plurality of clusters including a first cluster that comprises temporal-relation values of a cluster-subset of the second plurality of clusters with respect to a time instance of a reference cluster of the cluster-subset, corresponding to τ having a second value;
establishes a second layer comprising a first plurality of clusters of the second layer feature vectors;
establishes at least one successive layer after the second layer, each of the at least one successive layer corresponding to τ having a respective value; and
determines that a type of motion performed by the subject corresponds to one or more clusters of layer feature vectors of one of the at least one successive layer or the second layer.

44. The non-transitory computer-readable medium of claim 43, wherein the program, when executed by the processor,
detects a set of spike trains from the plurality neurons;
determines a first cluster based on the set of spike trains is consistent with the one or more clusters of layer feature vectors; and
determines whether the subject is intending or implementing the type of motion by determining that the first cluster is consistent with the one or more clusters of layer feature vectors.

45.-49. (canceled)
